(19) United States
(12) Patent Application Publication
(10) Pub. No.: US 2024/0358422 A1
(43) Pub. Date: Oct. 31, 2024

(54) GOUGER GRASPER DEVICE

(71) Applicant: Shukla Medical, St. Petersburg, FL (US)

(72) Inventor: Dustin Saxon, St. Petersburg, FL (US)

(73) Assignee: Shukla Medical, St. Petersburg, FL (US)

(21) Appl. No.: 18/643,298

(22) Filed: Apr. 23, 2024

Related U.S. Application Data
(60) Provisional application No. 63/498,547, filed on Apr. 27, 2023.

(51) Int. Cl. A61B 17/88; A61B 17/00; A61B 17/16; A61B 17/28

(52) U.S. Cl. CPC A61B 17/8847; A61B 17/1604; A61B 17/2816; A61B 17/2841; A61B 2017/00407; A61B 2017/2808

(57) ABSTRACT

A gouger grasper device including a handle at a proximal end of the device, the handle having a base and a trigger movably connected to the base, a chisel at a distal end of the device, a jaw at a distal end of the device and movable relative to the chisel, and a ratcheting assembly for moving the jaw relative to the chisel between an open position and a clamping position in response to movement of the trigger relative to the base.

### CROSS-REFERENCE TO RELATED APPLICATION

This application claims the benefit under 35 U.S.C. § 119 (e) to U.S. Provisional Application No. 63/498,547, filed Apr. 27, 2023, and entitled “Cement Gouger and Grasper Device,” the entire disclosure of which is hereby incorporated by reference for all purposes.

### BACKGROUND

During surgical procedures, bone cement is often used to secure surgical implants to bone or for other surgical purposes. However, the bone cement may need to be removed in later procedures where the surgical implant is removed/replaced or the surgical procedure is revised. Typical bone cement removal requires the use of two distinct tools-a chisel device or other sharp instrument to break the bone cement into pieces and a grasper device to remove the fragments of bone cement from the patient. Switching tools during a surgical procedure is time consuming and can lead to errors during removal and placement of the different tools.

### SUMMARY OF THE DISCLOSURE

According to an exemplary embodiment, the subject disclosure provides a gouger grasper device comprising a handle that includes a base and a trigger movably connected to the base, and a sheath extending from the handle. The device further comprises a chisel at a distal end of the sheath; a jaw at a distal end of the sheath and movable relative to the chisel; and a ratcheting assembly for moving the jaw relative to the chisel between an open position and a clamping position in response to movement of the trigger relative to the base.

According to an aspect, the trigger is pivotably connected to the base. According to another aspect, the ratcheting assembly comprises a ratchet arm having a plurality of teeth and connected to the base portion, and a button carried by the movable portion for engaging the teeth of the ratchet arm. According to another aspect, the ratcheting assembly further comprises an inner arm received in the base and configured to bias the trigger into an open position. According to another aspect, the inner arm and the trigger each define an aperture to allow passage of the ratchet arm therethrough.

According to an aspect, the ratcheting assembly further comprises, a connecting link connected to a distal end of the inner arm, a push rod connected to a distal end of the connecting link, and a linkage connected to a distal end of the push rod. According to another aspect, the jaw is connected to the linkage. According to another aspect, the linkage comprises a pair of first links and a pair of second links, wherein each of the first links has a proximal end coupled to the push rod and a distal portion coupled to the second links, and wherein the second links are pivotably coupled to the first links at their proximal ends and pivotably coupled to the jaw at their distal ends. According to another aspect, the device further comprises a biasing member disposed between a proximal end of the inner arm and the base for biasing the trigger into an open position.

Other features and advantages of the subject disclosure will be apparent from the following more detailed description of the exemplary embodiments.

### BRIEF DESCRIPTION OF THE SEVERAL VIEWS OF THE DRAWINGS

The foregoing summary, as well as the following detailed description of the exemplary embodiments of the subject disclosure, will be better understood when read in conjunction with the appended drawings. For the purpose of illustrating the present disclosure, there are shown in the drawings exemplary embodiments. It should be understood, however, that the subject application is not limited to the precise arrangements and instrumentalities shown.

FIG. 1 is an elevational view of a gouger grasper device in accordance with an exemplary embodiment of the subject disclosure;

FIG. 2 is a partial, elevational cross-sectional view of the gouger grasper device of FIG. 1;

FIG. 3 is an enlarged view of a handle of the gouger grasper device of FIG. 1;

FIG. 4 is a perspective view of a base of the handle of the gouger grasper device of FIG. 1;

FIG. 5 is a rear view of the base of the handle of FIG. 4;

FIG. 6 is a perspective view, with certain elements omitted for clarity of illustration, of a base of the handle and a ratcheting assembly of the gouger grasper device of FIG. 1;

FIGS. 7A and 7B are perspective views of a movable trigger of the handle of the gouger grasper device of FIG. 1;

FIG. 8 is a side elevational view of a chisel of the gouger grasper device of FIG. 1;

FIG. 9 is a front elevational view of the chisel of FIG. 8;

FIG. 10 is a perspective view of the chisel of FIG. 8;

FIG. 11 is a top plan view of the chisel of FIG. 8;

FIG. 12 is a front perspective view of a jaw of the gouger grasper device of FIG. 1;

FIG. 13A is a perspective view of the jaw and linkage of the gouger grasper device of FIG. 1;

FIG. 13B is an elevational view of the distal end of the gouger grasper device of FIG. 1 with the jaw shown in an open position with respect to the chisel;

FIG. 14A is a perspective view, with certain elements omitted for clarity of illustration, of a ratcheting assembly of the gouger grasper device of FIG. 1;

FIG. 14B is a perspective view of a button of the ratcheting assembly of FIG. 14A;

FIG. 15 is a perspective view of a connecting link of the gouger grasper device of FIG. 1; and

FIG. 16 is a perspective view of a push rod of the gouger grasper device of FIG. 1.

### DETAILED DESCRIPTION

Reference will now be made in detail to the various examples of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout this disclosure, various aspects of the subject disclosure can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the present disclosure can be practiced without one or more of the specific features or advantages of a particular exemplary embodiment. In other instances, additional features and advantages may be recognized in certain embodiments that may not be present in all exemplary embodiments of the subject disclosure.

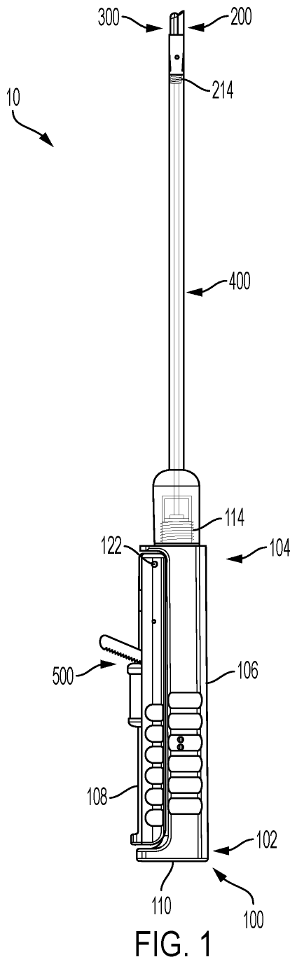

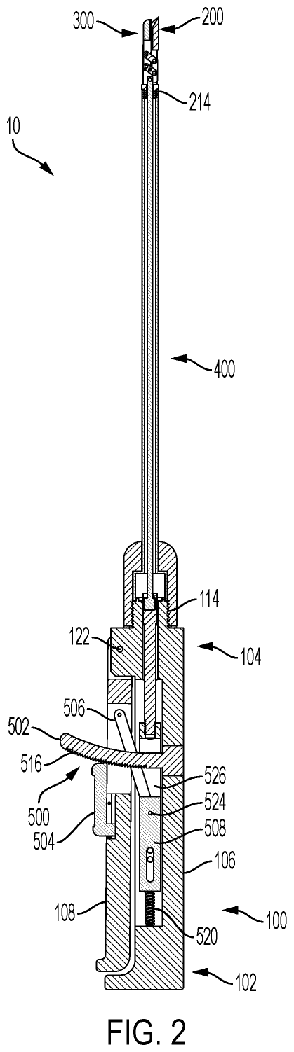

In accordance with an exemplary embodiment, the subject disclosure provides a gouger grasper device 10 configured as shown in FIGS. 1 and 2. The gouger grasper device 10 allows a user to both gouge bone cement and grasp broken cement pieces with a single device. The gouger grasper device 10 includes a handle 100, a chisel 200, a jaw 300, a sheath 400, and a ratcheting assembly 500.

The handle 100 is configured as best shown in FIGS. 1-7 and has a proximal end 102 and a distal end 104 opposite the proximal end. The handle is generally sized and shaped to be engaged by a user's hand and includes a base 106 and a trigger or moving portion 108. The handle also includes an impact surface 110 at its most proximal end. The impact surface 110 is configured to be struck by an unillustrated hammer or other striking device to assist in providing a force to the distal end of the device for impacting bone cement. The impact surface 110 is configured as planar surface and comprised of hardened material, such as a metal e.g., steel or iron, a ceramic, and the like, to withstand the striking force exerted by the striking device.

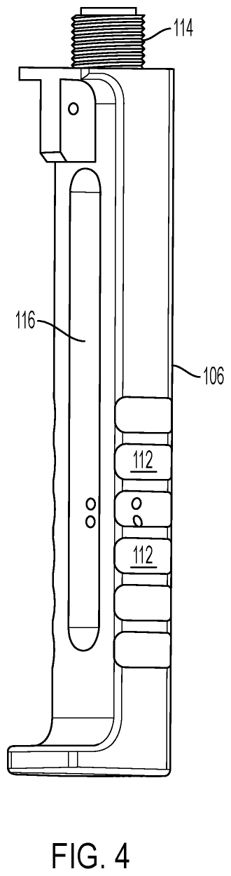

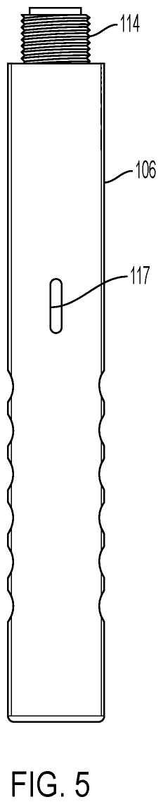

The base 106 is generally rectangular prismoidal in shape and has indentations 112 configured to provide a textured surface for enhancing a user's grasp of the device. At a distal end of the base 106 is a coupling 114 for connecting additional components to the base. The coupling 114 can be configured as a threaded sleeve or boss. Referring to FIGS. 4 and 5, the base 106 also includes a slotted recess 116 configured to allow the various ratcheting assembly components to be disposed or received at least partially within the base, and a slotted through hole 117 for receiving or seating one of the various ratcheting assembly components therein.

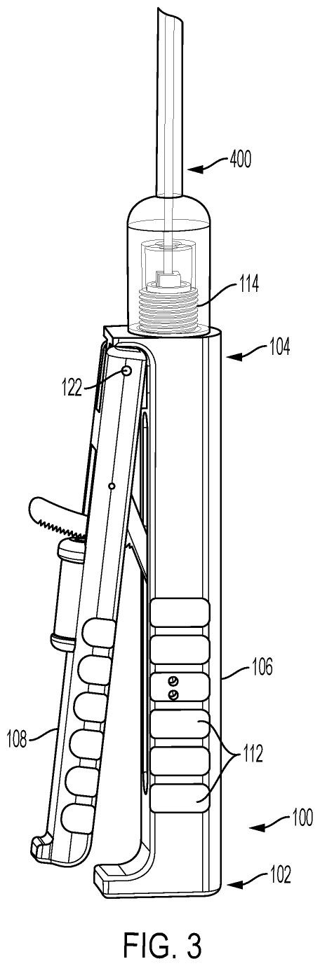

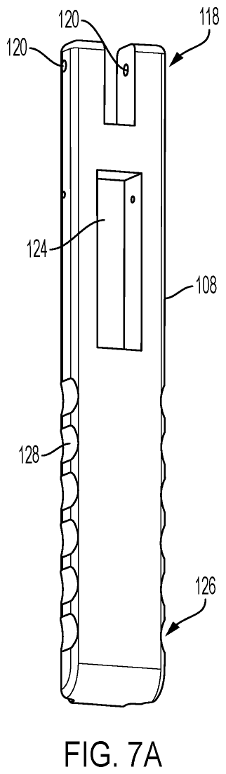

The trigger 108 is movably connected to the base 106. FIGS. 2 and 3 show the trigger 108 coupled to the base 106 near the distal end of the handle 100 with the proximal end of the trigger being a free end unattached to the base. The trigger 108 is generally configured as best shown in FIGS. 7A and 7B, having a generally rectangular shape including a bifurcated top or distal region 118 having aligned through holes 120 for receiving a pivot pin 122 (FIGS. 1-3) for pivotably connecting the trigger to the base. An aperture 124 is positioned about the midportion of the trigger forming a through hole. The trigger also includes a T-slot 125 adjacent to and in communication with the aperture 124.

The trigger is configured to rotate or move between an open handle position (FIG. 3) and a closed handle position (FIGS. 1 and 2) about the pivot pin 122. In the open handle position, the free (proximal) end of the trigger is spaced away from the base and in the closed handle position, the proximal end is positioned immediately adjacent the base.

The trigger also has a bottom or proximal end 126 with a plurality of indentations 128 configured to provide a surface for enhancing a user's grasp of the device. The indentations are preferably aligned along each side edge of the trigger.

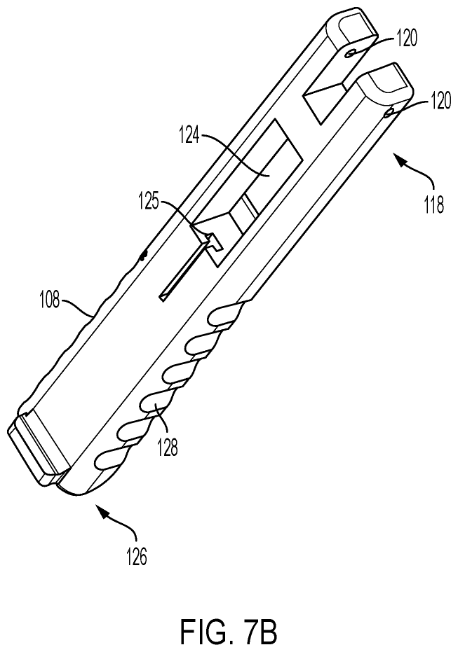

The chisel 200 is disposed or positioned about a distal end of the gouger grasper device 10 and configured as best shown in FIGS. 8-11. The chisel 200 includes a base 202 and a blade 204 extending from the base. A longitudinally extending through hole 205 is provided in the base, the purpose of which is described below. The blade 204 is an elongated blade having a cutting surface 206 at its most distal end and an elongated body 208. The elongated body 208 extends from the base 202, and more particularly extends asymmetrically from the base. In other words, the elongated body extends from one side of the base such that a longitudinal axis of the elongated body is offset from a longitudinal axis of the base.

The cutting surface 206 may be comprised of diamond or other materials which provide sufficient strength to cut bone cement. According to an aspect, the cutting surface 206 can be a tapered surface, a beveled edge, a pointed surface, a dual edge surface, conical shaped, and the like to assist with cutting bone cement.

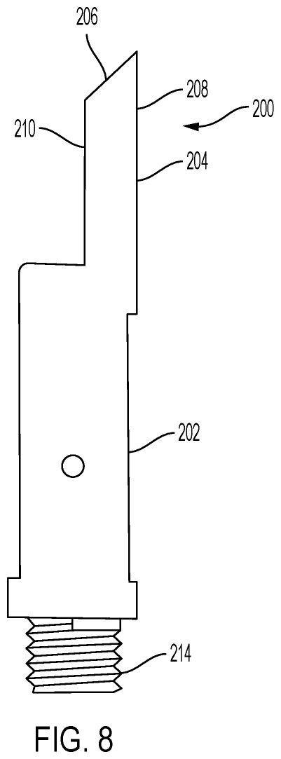

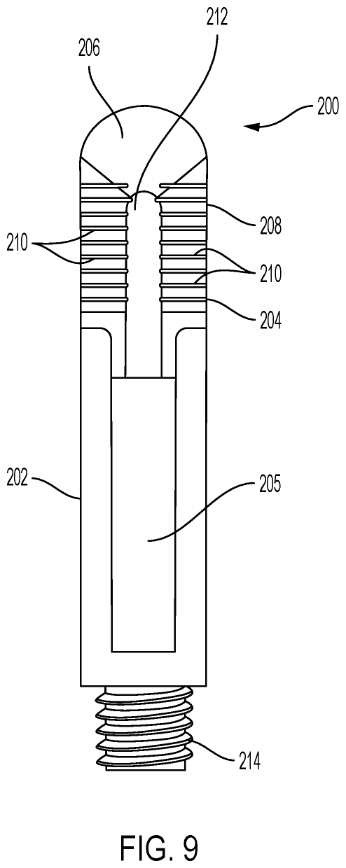

The chisel 200 also includes teeth, knurling or other surface engaging structures 210 disposed along a surface of the elongated body 208. Specifically, the teeth are positioned along a medial surface of the elongated body, as best shown in FIGS. 8-11. Additionally, in the example shown in FIGS. 9 and 10, the blade 204 may also include a center indentation or recess 212 which separates the teeth into two columns for providing additional gripping control of bone cement fragments.

A proximal end of the chisel base 202 includes a coupling 214 for coupling the chisel to a distal end of the sheath 400, described in greater detail below. The coupling 214 may be a threaded boss as illustrated in the figures or any other coupling or fastener known in the art and suitable for its intended purpose. The coupling 214 also includes an axially extending through hole 216 (FIG. 10) in communication with an internal portion of the chisel for the passage of other components to be disposed therethrough, as further described below.

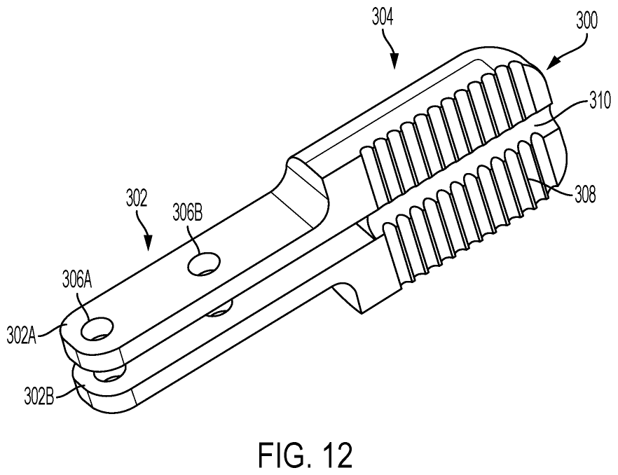

The gouger grasper device 10 also includes a jaw 300 configured as best shown in FIG. 12. The jaw 300 includes a securing portion 302 and an engagement portion 304. The securing portion 302 is coupled to the chisel and includes a pair of coupling bars 302A and 302B extending from the engagement portion. The coupling bars may include one or more apertures 306A, 306B to facilitate operative coupling to the chisel.

The engagement portion is of a generally rectangular box shaped configuration having a smooth outer surface and an inner surface that includes teeth, knurling or other surface engaging structures 308. In the example shown, the teeth 308 extend substantially horizontally across the inner surface. Additionally, the engagement portion may also include a center indentation or recess 310 which separates two columns of teeth for providing additional gripping control of bone cement fragments. An overall length of the engagement portion is configured to substantially match an overall length of the chisel such that the teeth of the chisel and the engagement portion of the jaw can substantially overlap when in a closed or clamped position, as further discussed below.

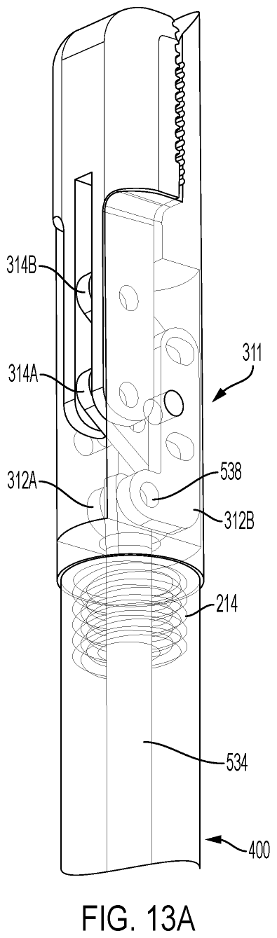

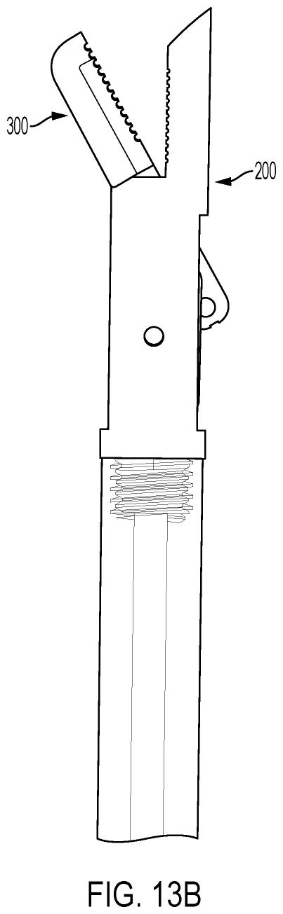

Referring to FIG. 13, the jaw 300 is movably coupled to the chisel 200 between an open position and a clamping position via a linkage 311 comprising a pair of first links 312A, 312B and a pair of second links 314A, 314B. The first links 312A, 312B are generally L-shaped links each having a proximal end coupled to a push rod 512 (described below) and a distal portion coupled to the second links 314A, 314B. The second links 314A, 314B are pivotably coupled to the first links 312A, 312B at their respective proximal ends and pivotably coupled to the apertures 306A, 306B of the jaw 300 (FIG. 12) at their respective distal ends. The first links 312A, 312B and the second links 314A, 314B are at least partially disposed in the aperture 205 (FIG. 9) of the chisel 200. The second links 314A, 314B are movable at an angle with respect to a longitudinal axis of the device 10 to move the jaw 300 outwardly (open, FIG. 13B) and inwardly (closed, FIG. 13A) relative to the chisel 200.

In the open position, the jaw 300 is disposed substantially spaced from the blade 204 such that the cutting surface of the chisel is free to gouge bone cement. That is, the jaw is both laterally spaced from the chisel and longitudinally spaced from the chisel. In the closed or clamping position (FIG. 13), the jaw 300 is disposed closer to the blade such that the teeth of the jaw 300 are configured to be substantially aligned with the plurality of teeth of the chisel to firmly grasp therebetween bits of cement dislodged by the chisel. In the open position, the tip of the chisel or blade is positioned further distally than a most distal end of the jaw. In particular, the entire length of the blade is positioned distally relative to the jaw when in the open position.

The ratcheting assembly 500 facilitates movement of the jaw 300 between the first position and the second position. More particularly, the ratcheting assembly operates to move the jaw relative to the chisel between the open position and the clamping position in response to movement of the movable trigger 108 relative to the base 106. As shown in FIGS. 2, 6 and 14A-16, the ratcheting assembly 500 includes a curved ratchet arm 502, a button 504, inner links 506, an inner arm 508, a connecting link 510 and a push rod 512.

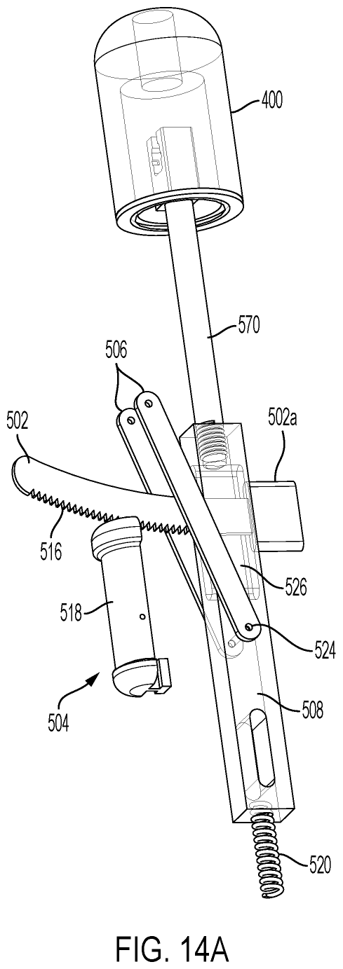

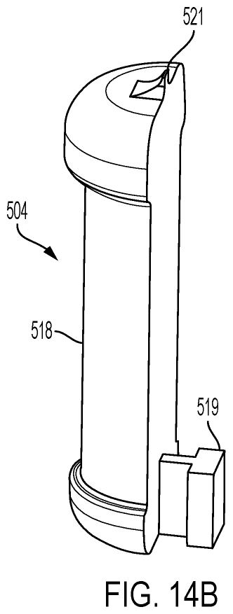

The ratchet arm 502 is an elongated arm having a base 502a (FIGS. 2 and 14A) disposed or seated within through hole 117 (FIG. 5) of the base 106, and extending through the aperture 124 of trigger 108 of the handle 100. The ratchet arm 502 has an arc length of about 10-45 degrees, but can be more or less including 5, 15, 20, 25, 30, 35, 40, and 50 degrees. The ratchet arm 502 also includes a plurality of teeth 516 substantially along an entire arc length of the elongated arm. The plurality of teeth 516 are configured to engage the button 504 to secure the ratchet arm 502 in a plurality of positions such as a first position, a second position, or between the first position and the second position throughout the arc length of the plurality of teeth.

The button 504 is configured as best shown in FIG. 14B and is movable between an activated position and a released position. The button 504 has a generally semi-cylindrical shaped body 518, a T-shaped guide 519 extending downwardly from the body for operatively engaging the T-slot 125 on the trigger, and a tapered nose 521 about a forward end of the body. The surface of the button is configured to be engaged by a user to move the button 504 between the activated position and the released position. In the activated position, the button 504 engages the teeth 516 of the ratchet arm 502 via the tapered nose and secures the trigger 108 of the handle 100 in a desired position. When it is desired for the ratchet arm 502 to be released, the user engages the button 504 and moves the button away from the teeth of the ratchet arm to disengage the tapered nose from the teeth of the ratchet arm and move to the released position. In the released position, the trigger returns to an open position under influence of a biasing member 520, discussed below.

The inner links 506 are coupled to the trigger 106 about one end and to the inner arm 508 about its opposite end. More particularly, the inner links straddle the ratchet arm 502 and are pivotably coupled at first ends thereof to the inner arm 508 and pivotably coupled at second ends thereof to the movable trigger 108.

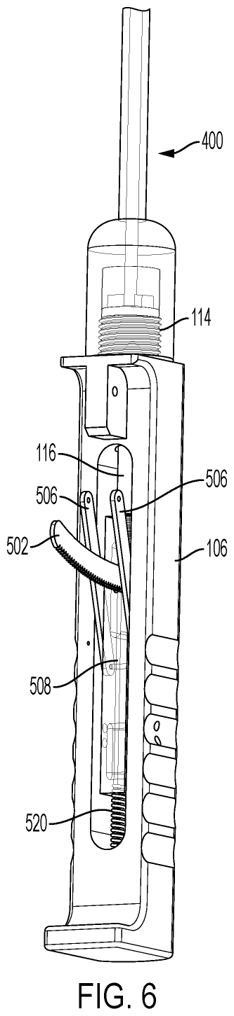

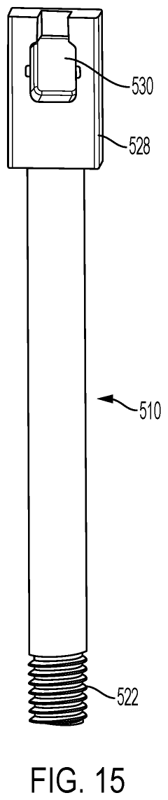

The inner arm 508 is generally rectangular prismoidal in shape and is disposed within the recess 116 (FIGS. 4 and 6) of the base 106 of the handle 100. As shown in FIGS. 2 and 6, a first end of the biasing member 520 is coupled to the proximal end of the inner arm and a second end of the biasing member abuts a distally facing surface of the recess 116. Constructed and arranged as such, the inner arm 508 is biased to move the trigger 108 into an open position. A distal end of the inner arm 508 may be internally threaded to allow coupling to a proximal externally threaded end 522 (FIG. 15) of the connecting link 510; however, various other coupling mechanisms may be applicable. The inner arm 508 includes one or more unillustrated apertures configured to allow the inner arm to be coupled via a pin 524 to the inner links 506 (FIG. 2). Additionally, the inner arm 508 includes an aperture 526 (FIG. 2) configured to allow the ratchet arm 502 to be disposed therethrough.

When the movable trigger 108 of the handle 100 is moved between the open position and the closed position, force is applied to the inner links 506 which is translated to the inner arm 508. Since the proximal end of the inner arm 508 is coupled to the biasing member 520, when squeezing force is applied to the trigger 108, corresponding force is applied to the inner arm by the inner links 506 whereby the biasing member is compressed which causes movement of the inner arm, the connecting link 510 and the push rod 512 in a proximal direction.

The connecting link 510 is best shown in FIG. 15 and is a generally cylindrical rod disposed at least partially within the distal end of the handle 100. As noted above, a proximal end of the connecting link 510 may be threaded at 522 to threadedly couple to a distal end of the inner arm 508. A distal end of the connecting link 510 includes an enlarged rectangular portion 528 extending from the generally cylindrical rod. The rectangular portion defines an indentation 530 configured to allow the connecting link to be coupled to the push rod 512. By way of example but not limitation, the indentation 530 is generally rectangular shaped and inset on one side of the rectangular portion 528.

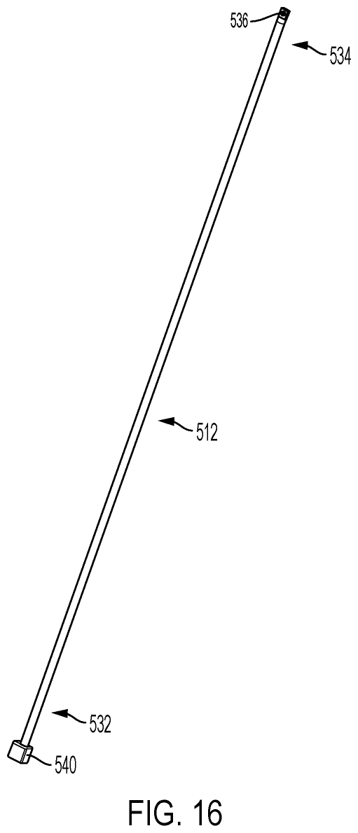

As shown in FIG. 16, the push rod 512 is a generally cylindrical rod having a proximal end 532 for coupling to the connecting link 510 and a distal end 534 including an aperture 536 for coupling to the pair of first links 312A, 312B via a pin 538 (FIG. 13). The proximal end of the push rod 512 includes an enlarged protrusion 540. The protrusion 540 may be generally rectangular in shape and is generally sized and shaped to be matingly seated within the indentation 530 of the connecting link 510, whereby the push rod is caused to move proximally and distally with the connecting link.

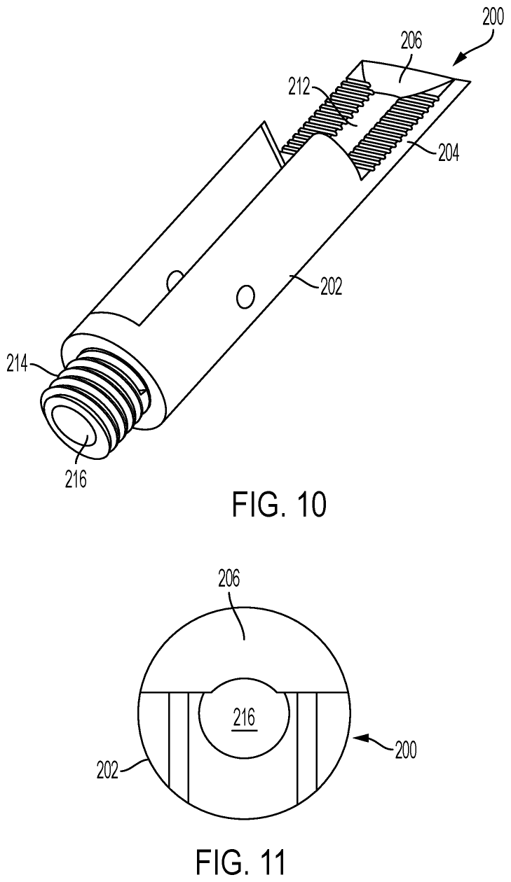

The gouger grasper device 10 further includes the aforementioned sheath 400 (FIGS. 1-3, 6 13 and 14A) extending over the connecting link 510 and the push rod 512 to protect the components from debris or other interferences. The sheath 400 is a rigid sheath and may be comprised of any material having a smooth surface. As shown in FIGS. 1-3, the proximal end of the sheath 400 can be internally threaded to receive the coupling 114 of the handle base 106. Similarly, as shown in FIGS. 1, 2 and 13, the distal end of the sheath 400 can be internally threaded to receive the coupling 214 at the proximal end of the chisel 200. As noted above, the coupling 214 desirably includes an axially extending through aperture 216 (FIGS. 10 and 11) through which the push rod 512 passes in order to enable its connection to the pair of first links 312A, 312B in the manner described above. Constructed and arranged as such, the chisel 200 may be releasably attached to the sheath 400, and the jaw 200 is configured to move toward and away from the chisel between the clamping and open positions.

In operation, a user inserts the gouger grasper device 10 into the patient with the movable trigger 106 biased to be swung outwardly into the open position with the cutting surface 206 of the chisel 200 exposed, whereby the user may use the device to break the desired bone cement. To assist with breaking the bone cement the user may employ a striking tool to provide an impact force to the proximal end 110 of the handle 100, if necessary. Once bone cement fragments are formed, the user will engage the movable trigger 108 and move it, such as by squeezing, towards the closed position. When the trigger is moved inwardly, the ratchet mechanism, i.e., the teeth 516 of the ratchet arm 502 and the distal end of the button 504 are activated to move the jaw 300 toward face-to-face alignment with the chisel 200 such that the jaw and chisel may act as a clamp to secure bone cement fragments therebetween. Once the bone cement fragments are secured between the jaw and the chisel, the user removes the device 10 from the patient and removes the bone cement fragments from the device. If additional usage is necessary, the user will move the button 504 thereby releasing the teeth 516 of the ratchet arm 502 and returns the jaw to its open position.

Having a gouger tool and a grasper tool in a single device eliminates the need for additional tools, saves time and prevents errors which might otherwise occur during removal and placement of separate gouger and grasper tools. As such, the gouger grasper device as described herein provides significant improvement and advantages over typical devices.

It will be appreciated by those skilled in the art that changes could be made to the various aspects described above without departing from the broad inventive concept thereof. It is to be understood, therefore, that the subject application is not limited to the particular aspects disclosed, but it is intended to cover modifications within the spirit and scope of the subject application as defined by the appended claims.

I/We claim:

1. A gouger grasper device comprising:
a handle that includes a base and a trigger movably connected to the base;
a sheath extending from the handle;
a chisel at a distal end of the sheath;
a jaw at a distal end of the sheath and movable relative to the chisel; and
a ratcheting assembly for moving the jaw relative to the chisel between an open position and a clamping position in response to movement of the trigger relative to the base.

2. The device of claim 1, wherein the trigger is pivotably connected to the base.

3. The device of claim 1, wherein the ratcheting assembly comprises:
a ratchet arm having a plurality of teeth and connected to the base portion; and
a button carried by the movable portion for engaging the teeth of the ratchet arm.

4. The device of claim 3, wherein the ratcheting assembly further comprises an inner arm received in the base and configured to bias the trigger into an open position.

5. The device of claim 4, wherein the inner arm and the trigger each define an aperture to allow passage of the ratchet arm therethrough.

6. The device of claim 4, wherein the ratcheting assembly further comprises:
a connecting link connected to a distal end of the inner arm;
a push rod connected to a distal end of the connecting link; and
a linkage connected to a distal end of the push rod.

7. The device of claim 6, wherein the jaw is connected to the linkage.

8. The device of claim 6, wherein the linkage comprises:
a pair of first links and a pair of second links,
wherein each of the first links has a proximal end coupled to the push rod and a distal portion coupled to the second links, and
wherein the second links are pivotably coupled to the first links at their proximal ends and pivotably coupled to the jaw at their distal ends.

9. The device of claim 4, further comprising a biasing member disposed between a proximal end of the inner arm and the base for biasing the trigger into an open position.
